(19) United States
(12) Patent Application Publication
(10) Pub. No.: US 2009/0004285 A1
(43) Pub. Date: Jan. 1, 2009

(54) Stable non-disintegrating dosage forms and method of making same

(76) Inventors: Liangping Yu, Mahwah, NJ (US); Paul H. Richardson, Vernon, NJ (US)

Correspondence Address: HOFFMANN & BARON, LLP, 6900 JERICHO TURNPIKE, SYOSSET, NY, 11791, US

(21) Appl. No.: 11/824,262

(22) Filed: Jun. 29, 2007

(51) Int. Cl. A61K 9/50; A61K 31/00; A61P 43/00

(52) U.S. Cl. 424/498; 514/789

(57) ABSTRACT

The present invention is a stable, non-disintegrable dosage form which combines the benefits of a microencapsulated substrate with the convenience of direct compression. The present invention is also directed to methods for producing directly compressed microencapsulated dosage forms to provide modified release and dosage form stability. The dosage unit can have a high active load.

### BACKGROUND OF THE INVENTION

The present invention is related to dosage forms and methods of making same, and, in particular, to stable non-disintegrable dosage units for delivery of active ingredients.

Over the years, the drug industry has sought to provide dosage forms which protect the pharmaceutically active and/or nutritional ingredients prior to consumption by a patient. Moreover, there is a need to deliver the bio-effecting agent in a manner and at a rate which benefits the patient.

U.S. Pat. No. 6,194,005 and U.S. Pat. No. 6,375,987, both to Farah, et al., discloses use of a lipid matrix agent composed of an alcohol ester of at least one fatty acid, to coat active with or without an adjuvant. They disclose use of a mixture of, for example, glycerol mono-, di-, and tri-behenate. A complex coating procedure and composition is required to tablet and to achieve desired release profile(s). The Farah, et al. dosage form is not stable and requires a maturation stage.

U.S. Pat. No. 5,690,959 to Palepu, et al., discloses preparation of a controlled release solid dosage form by a thermal infusion process used on a blend of the active ingredient and a hydrophobic waxy material, especially glyceryl behenate (a mixture of glycerol mono-, di-, and tribehenate), to provide agglomerates which must be sized before being formed into solid dosage forms. The process includes infusion heating on a preformed blend and a post-blend sizing before forming a dosage unit.

U.S. Pat. No. 7,052,706 to Mulye, discloses a process for preparing a sustained release pharmaceutical composition by blending a hydrophobic material, e.g., a mixture of glycerol mono-, di-, and tri-behenate, and forming a dosage unit. U.S. Pat. No. 4,590,062 and U.S. Pat. No. 4,894,236 also disclose blending of a hydrophobic material to form a dosage unit. U.S. Pat. No. 4,590,062 describes a method of direct compression which includes adding an active agent, a hydrophobic carbohydrate polymer, a wax and excipients to form a matrix upon direct compression. U.S. Pat. No. 4,894,236 describes a method of direct compression of a mixture of acetaminophen and a lipid. However, the processes set forth in the above references include blending a medicament and a hydrophobic material which is nonuniform and lacks the consistency desired for engineering and refining highly predictable release profiles in a patient.

U.S. Patent Application Publication No. US2004/008654A1 discloses a choline product and method of preparing same, which includes encapsulating a low hygroscopic choline salt with, inter alia, hydrogenated vegetable oil. However, the tablet is designed to immediately release the active ingredient found in the product, which is less than 50% of a unit dosage. Consequently the dosage form is designed for immediate release of active, even though encapsulates can release choline over time.

The art of producing dosage units for delivery of an active ingredient involves the integration of many disparate considerations. Ultimately, the dosage unit must be capable of delivering the active ingredient in such a manner as to successfully medically treat the patient who takes the dosage unit. Other important considerations include stabilizing the modified release profile over time, e.g., shelf life, and ease of manufacturing, producing dosage forms with a range of modified release profiles and low cost.

While various methods of direct compression and formulations formed by such processes have been developed, there remains a need to provide new direct compression methods which are economical, where the stability and integrity of the active agent, and the modified release dosage form, are maintained. Further there is a need for a dosage form that can provide modified release profiles at relatively high active loading. By using neutral lipid, spray coated microencapsulates directly compressed into tablets that are formulated not to disintegrate over the therapeutic range, these needs have been achieved.

### SUMMARY OF THE INVENTION

The present invention is a compressed non-disintegrable tablet and method of making same which includes a plurality of substrates that have been coated to provide microencapsulates. The coating material of the present invention is a neutral lipid-based coating and the substrates include at least one pharmacologically active ingredient. The coating material is spray coated onto the substrates such that the concentration of the active agent in the microencapsulate is at least about 50% by weight.

The microencapsulates are then directly compressed as in a tableting press in an amount of at least about 50% by weight of microencapsulate in each dose. The dosage unit resulting from the present invention is shelf stable and is non-disintegrable.

In other embodiments, the microencapsulates have at least about 60% by weight of active ingredient, or at least about 70%, or at least about 80%, and even at least about 90% by weight. And the dosage unit itself can have a level of microencapsulate in an amount of at least about 60% by weight, at least about 70% by weight, or at least about 80% by weight, and even at least about 90% by weight of microencapsulate.

The neutral lipids in the present invention includes triglycerides and waxes excluding monoglycerides and digylcerides. The terms “triglycerides” and “waxes” in the context of the present invention are defined and explained in the Detailed Description.

The triglycerides useful in the present invention can be selected from a group consisting of hydrogenated vegetable oils, hydrogenated animal fats, and combinations thereof. In one embodiment according to the present invention, the triglyceride is a hydrogenated vegetable oil selected from the group consisting of hydrogenated soybean oil, hydrogenated castor oil, hydrogenated cottonseed oil, hydrogenated canola oil, hydrogenated palm oil, and combinations thereof.

Waxes which can be used in the present invention can be selected from the group consisting of paraffin wax; a petroleum wax; a mineral wax such as ozokerite, ceresin, utah wax or montan wax; a vegetable wax such as, for example, carnauba wax, candelilla wax, Japan wax, bayberry wax or flax wax; an animal wax such as, for example, spermaceti; or an insect wax such as beeswax, Chinese wax or shellac wax, and combinations thereof.

The coating material of the present invention can also include a hydrophobic material. This hydrophobic material can be an active or a hydrophobic polymer such as ethylcellulose.

A preferred method of spray coating the substrates in accordance with the present invention is by fluidized bed coating. Other spray coating techniques include pan coating and any other spray coating methods. The neutral lipid-based coating material itself can include an active agent prior to coating the substrates.

The active agents useful in the present invention can be selected from the group consisting of antihistamines; antibiotics; antituberculosis agents; cholinergic agents; antimuscarinics; sympathomimetics; sympatholytic agents; miscellaneous autonomic drugs; iron preparations; haemostatics; cardiac drugs; antihypertensive agents; vasodilators; non-steroidal anti-inflammatory agents; opiate agonists; anticonvulsants; tranquilizers; chemotherapeutic agents; lipid lowering agents; H₂-antagonists; anti-coagulant and anti-platelet agents; bronchodilators; stimulants; barbiturates; sedatives; expectorants; antiemetics; gastro-intestinal drugs; antithyroid agents; genitourinary smooth muscle relaxants; vitamins; minerals; amino acids; herbal agents; botanical agents; enzymes; unclassified agents; diabetes agents; steroids; glucocorticoids; antivirals; antifungals; antiparasitic agents; antidiabetic agents; and any combinations or mixtures of the foregoing.

A particularly important group of active ingredients useful in the present invention can be taken from the group consisting of acetaminophen, caffeine, guaifenesin, an opioid analgesic, ranitidine, vitamin C, potassium chloride, niacin, anti-flush agents, lipid-lowering statins, and combinations thereof.

In one embodiment the active ingredient is guaifenesin which can be included in an amount of at least about 55% by weight of the dosage unit. In other embodiments, guaifenesin can be included in an amount of at least about 65%, and even at least about 75% by weight of the dosage unit.

In another embodiment, the active agent is niacin. Niacin can be included in an amount of at least about 50% by weight of said dosage unit. In other embodiments, niacin can be included in an amount of at least about 60%, at least about 70%, and even at least about 80% by weight of said dosage unit.

Another preferred embodiment of the present invention includes an active ingredient which is niacin and at least one other lipid-lowering statin. Another combination with niacin as an active ingredient is at least one anti-flush agent. Yet another combination with niacin as an active ingredient is fenofibrate.

Yet another preferred active agent can be an opioid analgesic, especially one selected from group consisting of codeine, hydromorphone, hydrocodone, oxycodone, morphine, meperidine and combinations thereof.

In an another embodiment the active agent is acetaminophen, and preferably included at a level at least about 50% of the microencapsulate. In other embodiments, acetaminophen can be included in an amount of at least about 60%, at least about 70%, and even at least about 80% by weight of said dosage unit.

In other embodiments, a plurality of microencapsulated substrates may be directly compressed into a tablet substantially without the aid of excipients (i.e., the dosage form contains no additional excipients or only negligible amounts of the same).

In other embodiments, a plurality of microencapsulated substrates may be directly compressed into a tablet with the aid of at least one excipient. The number of substrates included in a unit dose (e.g., a single directly compressed tablet) will be such that the total amount of active agent incorporated therein is sufficient to provide the desired effect (e.g., in the case where the active agent is a drug, a desired therapeutic effect). Each excipient may be any excipient suitable for use in direct compression techniques as long as the dosage form does not disintegrate over the therapeutic range. In certain embodiments, the excipient may be a pre-manufactured direct compression excipient.

Excipient(s) can be included in an amount not to exceed 50% by weight of the dosage unit, and, in separate embodiments, not greater than 40%, than 30%, 20%, 10% and 5% respectively. The excipient(s) can be selected from flavoring agents, acidifiers, sweetners, taste-makers, lubricants, and combinations thereof. Specific examples of excipients useful in the present invention include, but are not limited to, an ingredient selected from the group consisting of sucrose, dextrose, lactose, microcrystalline cellulose, xylitol, fructose, polyethylene glycol (PEG), silicified microcrystalline cellulose, maltodextrin, sorbitol, and combinations thereof.

The active dosage unit active agent dissolves from the active dosage form in the absence of disintegration during dissolution of active within the therapeutic range.

In one embodiment, the active agent is guaifenesin and the dosage unit is designed to release from about 10% to about 35% of the guaifenesin after one (1) hour and from about 50% to about 90% after eight (8) hours in vitro.

In another embodiment, the active agent is niacin in a one a day dosage unit and the dosage unit it designed to release from about 5% to about 30% of the niacin after one (1) hour and from about 35% to about 70% after eight (8) hours in vitro. In yet another embodiment, the active agent is niacin in a two a day dosage unit and the dosage unit is designed to release from about 5% to about 40% of the niacin after one (1) hour and from about 25% to about 75% after four (4) hours in vitro.

The direct compressed dosage unit of the present invention provides a modified release. In one embodiment, the active dosage unit active agent has an “early and sustained” modified release. In other embodiments, the active dosage unit active agent has one of the following release profiles: delayed, controlled, extended, site specific, slow, pulsatile, or combinations thereof.

The direct compressed dosage forms of the present invention are suitable for providing a direct compressed dosage form that allows for a high load of active agent and/or the use of low levels of excipients. The microencapsulated substrates of the present invention may be used to prepare a direct compressed dosage form with enhanced physical properties, e.g., stability, better binding during compression, etc., which in turn may also provide a direct compressed dosage form with a high level (load) of active agent as compared to other art known direct compressed dosage forms.

The invention is also directed to a solid dosage form, having a plurality of microencapsulated substrates which include at least about 50% active agent, said substrates microencapsulated in a neutral lipid-based coating.

In each of the embodiments of the invention, the dosage form can further include one or more additional active agent(s). Such additional active agent(s) may for example be (i) included with said substrates; (ii) mixed with said microencapsulated substrates prior to compression; (iii) coated onto the surface of the compressed solid dosage form; and (iv) included in the coating of the microencapsulates.

In various embodiments, the substrates may be (i) a granulate comprising active agent and one or more pharmaceutically acceptable diluents; (ii) active agent coated onto the surface of inert beads; or (iii) pellets comprising active agent and one or more pharmaceutically acceptable diluents.

The present invention provides a unit dosage which does not disintegrate and whereby active ingredient dissolves from a neutral lipid matrix. As a result of dissolution from a dosage form prepared from a coated active substrate, the practitioner can precisely design coatings and dosage units with more predictable product quality and release profile. In many cases the requirement for excipients is eliminated, but, in any event, certainly a significantly reduced about of excipient(s) can be used to achieve desired product characteristics and release profile.

As a result of the present invention a solid dosage form is provided which can be prepared by direct compression of microencapsulated substrates. As a result of the process and the composition of the microencapsulates, they can be easily guided into a direct compression device such as a tablet compaction apparatus without the requirement for additional flow agents.

Yet another advantage of the present invention is the ability to provide a selected release profile without the disadvantages associated with lipid-based disintegrable dosage forms, e.g., instability.

Another advantage of the present invention is that the coated substrates can be compressed into a dosage form using less of expensive excipients for manufacturing the final dosage units.

Yet another advantage is that the present invention can be made essentially without the need for excipients, but that excipients can be entered or added to the extent necessary to fine-tune different features of the dosage product.

For a better understanding of the present invention, together with other and further objects, reference is made to the following description, taken in conjunction with the accompanying drawings and its scope will be pointed out in the appended claims.

### BRIEF DESCRIPTION OF THE DRAWINGS

FIG. 1 is a graph showing a comparison of an in vivo release profile of a niacin de in accordance with the invention and a commercial product, Niaspan®.

FIG. 2 and FIG. 3 are graphs which depict correlation studies between an in vitro ported in Example VII and an in vivo study reported in FIG. 1, respectively.

### DETAILED DESCRIPTION OF THE INVENTION

In order that the invention describe herein may be further understood, the following definitions are provided for the purposes of the disclosure:

The term “active agent” or “active ingredient” or “bio-effecting” is defined as any compound that provides an effect in an environment of use. In certain embodiments, the effect is a therapeutic effect. An active agent may be any active pharmaceutical ingredient (API), a nutraceutical active agent, or an herbal remedy. An active ingredient may be a vaccine, or an antibiotics or prebiotics or probiotics.

The term “excipient” is defined as any pharmaceutically acceptable excipient suitable for animal, e.g., human, consumption.

The term “substrate” is defined as the active agent itself, the active agent combined together with at least one pharmaceutically acceptable excipient, bead, granule, pellet, spheroid, etc. like with the active agent contained therein or thereon.

The term “stable” or “shelf stable” in the context of the present invention means that the change in dissolution rate of an active agent from a unit dosage over the therapeutic range after being subjected to accelerated storage conditions is not greater than ±10%. For the purpose of testing stability of dosage units prepared in accordance with the present invention, accelerated storage conditions used herein were 40° C. at 75% relative humidity for three (3) months. Thus, if a dosage unit normally releases 30% active ingredient after four (4) hours of dissolution in vitro, but undergoes a change in dissolution rate of active after being subjected to accelerated storage conditions of not less than 20% active nor greater than 40% active after four (4) hours, it is considered “stable” or “shelf stable.”

For purposes of the present invention, the term “dissolve” means active agent solubilization.

For purposes of the present invention, the term “patient” is defined as a human or animal inflicted with a disease or condition that requires treatment with an active agent.

The term “coating” in the present invention means a method of encapsulating a substrate by spray coating. Fluidized bed coating is the most preferred method of spray coating. Others include pan coating and any other spray coating technique.

The term “neutral lipid-based coating” in the context of the present invention means a coating which primarily includes “neutral lipid(s).”

The term “neutral lipid” in the context of the present invention is defined as any triglyceride or wax, and combinations thereof, excluding monoglycerides and diglycerides.

The term “triglycerides” in the context of the present invention means a coating having substantially all triglycerides, e.g., at least about 95%-98%, and preferably about 97% triglyceride. “Neutral lipid-based coating” does not include lipid-based coatings which include mixtures of mono-, di-, and triglycerides such as, for example, COMPRITOL 888, a Gattefossé product. The triglycerides used herein include hydrogenated vegetable oils, hydrogenated animal fats, and combinations thereof. Other ingredients can be present in the coating, but not to the extent that the coating loses its unique triglyceride nature.

The term “waxes” in the context of the present invention is defined as esters of high molecular weight, even-numbered monohydric alcohols (C₁₆ to C₃₆) and fatty acids (C₁₄ to C₃₆).

The term “subject” is defined as a human or animal that does not have any disease or condition that requires treatment with an active agent, e.g., a normal volunteer.

The term “dosage form” is defined as dosage forms that are used orally, rectally, vaginally and may include, but are not limited to, tablets, caplets, minitablets, lozenges, sublingual tablets, buccal tablets, suppositories and the like.

“Therapeutic dissolution range” in the context of the present invention means the release of active ingredient(s) at the desired rate and over the desired time period for the selected treatment with the delivered active ingredient(s).

“Directly compressing” in the present invention means compaction compressing the powder formulation on a tablet press.

“One-component dosage unit” in the context of the present invention means the presence of only one combination or system of “active plus coating” to provide a desired release profile in a patient. Thus, for example, the early and sustained release of active ingredient/agent depends on the dissolution characteristics of the only “active plus coating” system compressed into the dosage unit.

“Disintegrable” (and “non-disintegrable”) in the context of the present invention means capable (or not capable) of breaking into parts or fragments during active dissolution. The dosage unit of the present invention relies solely on dissolution for delivery of active and does not disintegrate.

For purposes of the present invention, the term “modified release” is defined as release patterns which include “early and sustained”, delayed, controlled, extended, site specific, slow or pulsatile release or combination thereof.

The substrate to be microencapsulated may be the active agent itself, the active agent combined together with one or more excipients into suitably sized particles (granules), shaped into pellets, or manufactured as spheroids. In certain embodiments, the active agent itself is a granulate of acceptable size such that the coating can be directly applied onto its surface in an even manner to create a desirable microencapsulate. In other embodiments of the invention, the active agent is granulated (e.g., wet granulated) together with an excipient(s) to make desirable granules which can be coated. In such embodiments, the active agent is typically wet granulated with a diluent (e.g., lactose, sucrose, starch, and the like). Generally, the resultant granulate has a particle size ranging from about 0.01 to about 3 mm. In certain preferred embodiments, the active agent granulate is sieved and a fraction which is from about 0.1 mm to about 0.6 mm (and in certain embodiments less than 0.3 mm) is then separated and further processed via microencapsulation and direct compression into a final dosage form. Alternatively, the substrates used in the invention may comprise a pharmaceutically acceptable sugar sphere (bead) coated with the active agent. Sugar spheres are solid excipients which are composed of one or more of sugar, starch, cellulose, etc. and typically have a size ranging from about 0.3 mm to about 1.4 mm. Pellets are generally considered in the art to comprise small, sterile cylinders (e.g., about 3 mm in diameter by about 8 mm in length), which are formed from compression from a mass comprising active agent and one or more excipients. On the other hand, the substrate may comprise a matrix spheroid in which the active agent is incorporated together with the excipient(s) in a substantially uniform fashion. One skilled in the art will also appreciate that pharmaceutically acceptable excipients may be utilized in the preparation of such substrates without changing the basic character of the invention.

The present invention utilizes substrates comprising an active agent microencapsulated with a neutral lipid protective coating in the manufacture of a direct compressed dosage form. The invention includes the final dosage form which includes a plurality of microencapsulated substrates compressed into a solid dosage form. In certain embodiments explained in more detail in the following paragraphs, additional materials (including pharmaceutically acceptable excipients) may be incorporated into dosage unit, e.g., excipients may further be admixed with a plurality of microencapsulated substrates, and the combination compressed into a solid dosage form, as long as the dosage form does not disintegrate within the therapeutic dissolution range.

### Neutral Lipid Coating

In preferred embodiments of the invention, substrates comprising active agent(s) are coated with a protective neutral lipid-based coating. Neutral lipids include triglycerides and waxes, excluding monoglycerides and diglycerides.

Triglycerides suitable for use as a neutral lipid coating in the dosage forms of the invention are known to those skilled in the art, and basically hydrogenated vegetables oils and animal fats. Hydrogenated vegetable oils can include, but are not limited to, cashew, castor bean, linseed, grape seed, hemp seed, mustard seed, poppy seed, rape seed (canola oil), safflower, sesame seed, sunflower, almond, algae, apricot, argan, avocado, corn oil, cotton seed, coconut, fusarium, hazelnut, neem oil, palm, palm kernel, peanut, pumpkin, rice bran, walnut, soybean oil and any combinations or mixtures thereof.

The waxes can include the group consisting of paraffin wax; a petroleum wax; a mineral wax such as ozokerite, ceresin, utah wax or montan wax; a vegetable wax such as, for example, carnauba wax, Japan wax, bayberry wax or flax wax; an animal wax such as, for example, spermaceti; or an insect wax such as beeswax, Chinese wax or shellac wax, and combinations thereof.

A hydrophobic material can be included in the coating and can be selected from any alkylcellulose or other hydrophobic cellulosic materials and other hydrophobic materials, but most preferably ethylcellulose. In certain embodiments, part or all of the hydrophobic material may be the active agent itself.

The amount of hydrophobic material contained in the neutral lipid coating, when present at all, may range from about 0.1% to about 30%, from about 0.1% to about 20% or from about 0.1% to about 10%, of the total weight of neutral lipid coating.

One or more optional pharmaceutically acceptable excipients may also be included or dissolved in the neutral lipid coating. The excipient(s) can be included up to 50% of the dosage unit; in one embodiment not greater than 40%; in another embodiment not greater than 30%; in yet another embodiment not greater than 20%; and in another embodiment not greater than 10%; and finally, one embodiment includes excipients up to no greater than 5% by weight of the dosage unit.

Such optional excipients which may be included in the neutral lipid coating of the present invention include, but are not limited to, flavoring agents, taste-masking agents, bitter blockers, plasticizers, binders, sensory masking agents, flavors, materials that dissolve at different pHs, antioxidants, cellulose and cellulose derivatives, and the like. Other excipients suitable which may be included in the neutral lipid coating are well known to those skilled in the art and are described in the Handbook of Pharmaceutical Excipients, American Pharmaceutical Association (2005), incorporated by reference herein.

In certain embodiments of the present invention, the neutral lipid is a hydrogenated vegetable oil, e.g., soybean oil, and the hydrophobic material is ethylcellulose, wherein the amount of hydrogenated vegetable oil may range from about 99.9% to about 80% and the amount of ethylcellulose may range from about 0.1% to about 20%, by weight.

The method of applying the coating to the active agent is by spray coating. The active agent can be sprayed with the coatings of the present invention, the active agent being suspended by a flow of air (fluidized bed). U.S. Pat. Nos. 4,511,584 at columns 3-5 and 4,511,592 at column 4, the disclosures of which are incorporated herein by reference, teach preferred methods of applying coatings to granular particles. U.S. Pat. Nos. 4,537,784 at columns 4-4; 4,497,845 at column 4; U.S. Pat. Nos. 3,819,838; 3,341,446; 3,279,994; 3,159,874; 3,110,626; 3,015,128; 2,799,241; and 2,648,609, which disclosures are all incorporated herein by reference, teach additional methods and apparatus for applying coatings which may be used to produce the coated active agents used in the present invention.

### Modified Release Formulations

One skilled in the art will appreciate that the directly compressed substrate dosage forms of the present invention may be modified to alter the release of the active agent from the dosage form. Means to modify the release include, neutral lipid type, the amount of neutral lipid, the type and amount excipient, external dosage unit coatings, and combinations thereof as long as the dosage form does not disintegrate within the therapeutic dissolution range.

Modified releases are release patterns which include “early and sustained”, delayed, controlled, extended, site specific, slow, pulsatile, and combinations thereof.

Early and sustained release means that a single release profile of the active ingredient from a dosage unit prepared in accordance with the invention is such that (i) 80% active ingredient is released over four (4) hours or greater, and that (ii) the ratio of the “time at 80% release of active” to the “time at 25% release of active” is equal to or greater than eight (8):

$$\frac{Time of Release of 80 \% of Active}{Time of Release of 25 \% of Active} \geq 8$$

Thus, if it takes twelve (12) hours for a dosage unit to release 80% of active ingredient and one (1) hour to release 25%, the ratio would be 12 (i.e., 12 hrs/1 hr). If however, the time to release 25% active ingredient is two (2) hours, the ratio would be 6 (i.e., 12 hrs/2 hrs) and not be within the “early and sustained release efficacy” of the invention.

Once a microencapsulated substrate has been prepared, it may then be utilized for preparing the direct compressed dosage forms of the present invention. In certain embodiments, a plurality of the microencapsulated substrates may be directly compressed without any additional excipients, or only negligible amount(s) of additional excipient(s), to obtain a directly compressed dosage form. Thus, the use of the microencapsulated substrates described above may reduce the need for excessive excipients in the direct compressed dosage forms.

In other embodiments, a plurality of the microencapsulated substrates may be directly compressed together with one or more excipients. The excipient may be any pharmaceutically acceptable excipient suitable for direct compression techniques. In such embodiments, the excipient may include (but is not limited to) a diluent selected from, e.g., a monosaccharide, a disaccharide, a polyhydric alcohol, a sugar alcohol (e.g., mannitol), a cellulose (such as microcrystalline cellulose), and/or mixtures thereof. Examples of suitable diluents include sucrose, dextrose, lactose, microcrystalline cellulose, xylitol, fructose, sorbitol. Other suitable excipients for use in the direct compressed dosage forms of the present invention may also include pre-manufactured direct compression excipients. Examples of such pre-manufactured direct compression excipients include Prosolv® (silicified microcrystalline cellulose), Emcocel® (microcrystalline cellulose, N.F.), Emdex® (dextrates, N.F.), and Tab-Fine® (a number of direct-compression sugars including sucrose, fructose, and dextrose), all of which are commercially available from JRS Pharma Inc., Patterson, N.Y.). Other direct compression diluents include Anhydrous lactose (Lactose N.F., anhydrous direct tableting) from Sheffield Chemical, Union, N.J. 07083; Elcema® G-250 (Powdered cellulose, N.F.) from Degussa, D-600 Frankfurt (Main) Germany; Fast-Flo Lactose® (Lactose, N.F., spray dried) from Foremost Whey Products, Banaboo, Wis. 53913; Maltrin® (Agglomerated maltrodextrin) from Grain Processing Corp., Muscatine, Iowa 52761; Neosorb 60® (Sorbitol, N.F., direct-compression) from Roquette Corp., 645 5th Ave., New York, N.Y. 10022; Nu-Tab® (Compressible sugar, N.F.) from Ingredient Technology, Inc., Pennsauken, N.J. 08110; Other excipients which may be admixed together with a plurality of microencapsulated substrates and directly compressed into a solid dosage form include cellulose derivatives, such as hydroxypropylmethylcellulose (HPMC), flavoring agents, acidifiers, sweeteners, taste-maskers, lubricants (e.g., magnesium stearate, stearic acid) and any combinations or mixtures of the foregoing. These excipients can be included as long as the dosage form does not disintegrate over the therapeutic range.

The excipients utilized preferably have uniform packing characteristics over a range of different particle size distributions and are capable of processing into the final composition (e.g., tablets) using direct compression techniques.

In certain embodiments, the amount of excipient admixed with the microencapsulated substrates prior to direct compression may range from about 0% to about 50%. However, particularly in applications where a high percentage of the microencapsulate in the dosage form is desired, the amount of excipient admixed with the microencapsulated substrates prior to direct compression can range from about 0% to about 30%. In certain more preferred embodiments where the final dosage form comprises a high load of active agent, the amount of excipient in the dosage form may range from about 0% to about 20%, from about 0% to about 15%, or from about 0% to about 10%, 0% to about 5%.

Those skilled in the art will recognize that changes in the physical properties of the dosage form such as surface area, size, shape, density and porosity, are other means to modify the release of the active agent.

In certain embodiments, the dosage form for guaifenesin according to the present invention provides a release profile such that a dissolution rate results, in vitro, when measured using USP dissolution method #2, Varian VK 7000 instrument, 900 ml aqueous fill, 50 rpm paddle speed 37° C., of 10%-35% (by weight) of active released after one (1) hour; and between 50% and 90% of active release after eight (8) hours.

In yet another embodiment, the one a day dosage form for niacin provides a release profile such that a dissolution rate results, in vitro, when measured using USP dissolution method #2, Varian VK 7000 instrument, 900 ml aqueous fill, 50 rpm paddle speed 37° C., of 5%-30% (by weight) of active released after one (1) hour; and between 35%-70% of active release after eight (8) hours. Whereas, the two a day dosage form for niacin provides a release profile such that a dissolution rate results, in vitro, when measured using USP dissolution method #2, Varian VK 7000 instrument, 900 ml aqueous fill, 50 rpm paddle speed 37° C., of 5%-40% (by weight) of active released after one (1) hour; and between 25%-75% of active release after four (4) hours.

### Active Agents

Active agents suitable for use in the present invention may include, but are not limited to, water soluble and water insoluble agents. In certain embodiments, the active agent may be a heat-labile active agent.

### Drugs

Examples of active agents that are suitable for incorporation in the present invention include drugs such as: antihistamines (e.g., azatadine maleate, brompheniramine maleate, carbinoxamine maleate, chlorpheniramine maleate, dexchlorpheniramine maleate, diphenhydramine hydrochloride, doxylamine succinate, methdilazine hydrochloride, promethazine, trimeprazine tartrate, tripelennamine citrate, tripelennamine hydrochloride and triprolidine hydrochloride); antibiotics (e.g., penicillin V potassium, cloxacillin sodium, dicloxacillin sodium, nafcillin sodium, oxacillin sodium, carbenicillin indanyl sodium, oxytetracycline hydrochloride, tetracycline hydrochloride, clindamycin phosphate, clindamycin hydrochloride, clindamycin palmitate HCL, lincomycin HCL, novobiocin sodium, nitrofurantoin sodium, metronidazole hydrochloride); antituberculosis agents (e.g., isoniazid); cholinergic agents (e.g., ambenonium chloride, bethanecol chloride, neostigmine bromide, pyridostigmine bromide); antimuscarinics (e.g., anisotropine methylbromide, clidinium bromide, dicyclomine hydrochloride, glycopyrrolate, hexocyclium methylsulfate, homatropine methylbromide, hyoscyamine sulfate, methantheline bromide, hyoscine hydrobromide, oxyphenonium bromide, propantheline bromide, tridihexethyl chloride); sympathomimetics (e.g., bitolterol mesylate, ephedrine, ephedrine hydrochloride, ephedrine sulphate, orciprenaline sulphate, phenylpropanolamine hydrochloride, pseudoephedrine hydrochloride, ritodrine hydrochloride, salbutamol sulphate, terbutaline sulphate); sympatholytic agents (e.g., phenoxybenzamine hydrochloride); miscellaneous autonomic drugs (e.g., nicotine); iron preparations (e.g., ferrous gluconate, ferrous sulphate); haemostatics (e.g., aminocaproic acid); cardiac drugs (e.g., acebutolol hydrochloride, diisopyramide phosphate, flecainide acetate, procainamide hydrochloride, propranolol hydrochloride, quinidine gluconate, timolol maleate, tocainide hydrochloride, verapamil hydrochloride); antihypertensive agents (e.g., captopril, clonidine hydrochloride, hydralazine hydrochloride, mecamylamine hydrochloride, metoprolol tartrate); vasodilators (e.g., papaverine hydrochloride); non-steroidal anti-inflammatory agents (e.g., choline salicylate, ibuprofen, ketoprofen, magnesium salicylate, meclofenamate sodium, naproxen sodium, tolmetin sodium); opiate agonists (e.g., codeine hydrochloride, codeine phosphate, codeine sulphate, dextromoramide tartrate, hydrocodone bitartrate, hydromorphone hydrochloride, pethidine hydrochloride, methadone hydrochloride, morphine sulphate, morphine acetate, morphine lactate, morphine meconate, morphine nitrate, morphine monobasic phosphate, morphine tartrate, morphine valerate, morphine hydrobromide, morphine hydrochloride, propoxyphene hydrochloride); anticonvulsants (e.g., phenobarbital sodium, phenytoin sodium, troxidone, ethosuximide, valproate sodium); tranquilizers (e.g., acetophenazine maleate, chlorpromazine hydrochloride, fluphenazine hydrochloride, prochlorperazine edisylate, promethazine hydrochloride, thioridazine hydrochloride, trifluoroperazine hydrochloride, lithium citrate, molindone hydrochloride, thiothixine hydrochloride); chemotherapeutic agents (e.g., doxorubicin, cisplatin, floxuridine, methotrexate, combinations thereof, etc.); lipid lowering agents (e.g., gemfibrozil, clofibrate, niacin, fenofibrates, HMG-CoA reductase inhibitors, such as for example, atorvastatin, cerivastatin, fluvastatin, lovastatin, pravastatin, simvastatin, etc.); H₂-antagonists (e.g., cimetidine, famotidine, nizatidine, ranitidine HCl, etc.); anti-coagulant and anti-platelet agents (e.g., warfarin, cipyridamole, ticlopidine, etc.); bronchodilators (e.g., albuterol, isoproterenol, metaproterenol, terbutaline, etc.); stimulants (e.g., benzamphetamine hydrochloride, dextroamphetamine sulphate, dextroamphetamine phosphate, diethylpropion hydrochloride, fenfluramine hydrochloride, methamphetamine hydrochloride, methylphenidate hydrochloride, phendimetrazine tartrate, phenmetrazine hydrochloride, caffeine citrate); barbiturates (e.g., amylobarbital sodium, butabarbital sodium, secobarbital sodium); sedatives (e.g., hydroxyzine hydrochloride, methprylon); expectorants (e.g., potassium iodide, guaifenesin); antiemetics (e.g., benzaquinamide hydrochloride, metoclopropamide hydrochloride, trimethobenzamide hydrochloride); gastro-intestinal drugs (e.g., ranitidine hydrochloride); heavy metal antagonists (e.g., penicillamine, penicillamine hydrochloride); antithyroid agents (e.g., methimazole); genitourinary smooth muscle relaxants (e.g., flavoxate hydrochloride, oxybutynin hydrochloride); vitamins (e.g., thiamine hydrochloride, ascorbic acid); unclassified agents (e.g., amantadine hydrochloride, colchicine, etidronate disodium, leucovorin calcium, methylene blue, potassium chloride, pralidoxime chloride; steroids, particularly glucocorticoids (e.g., prednisolone, prednisone, cortisone, hydrocortisone, methylprednisolone, betamethasone, dexamethasone, triamcinolone); antiviral agents (e.g., vidarabine, acyclovir, ribavirin, amantadine hydrochloride, interferons, dideoxyuridine), antifungal agents (e.g., nystatin, miconazole, tolnaftate, undecyclic acid and its salts); antiparasitic agents (e.g., quinacrine, chloroquine, and quinine); diabetes agents (insulin, metformin, alpha-lipoic acid, glucose); and any combinations or mixtures of the foregoing.

Although a particular benefit of the present invention concerns the ability to incorporate a high load of active agent(s) into the dosage form (by virtue of the neutral lipid coating of the substrate comprising the active agent), it is possible to include a wide range of amounts of active agent(s) relative to the total weight of the dosage forms of the invention. The load of the active agent in the microencapsulate is at least about 50%. In other embodiments, the load is at least about 60%. In certain embodiments, the load of active ingredient is at least about 70%, in another embodiment, at least about 80%, and in yet another embodiment, at least about 90%.

Guaifenesin dosage forms of the present invention provide a load of microencapsulates of guaifenesin sufficient to provide at least about 55% guaifenesin, at least about 65%, and, in certain embodiments, at least about 75%.

When the active agent utilized in the present invention is acetaminophen, the microencapsulates of acetaminophen are sufficient to provide at least about 50% acetaminophen, preferably at least about 60%, and, in other more preferred embodiments, at least about 70% and at least about 80% in the final dosage form.

When the active agent utilized in the present invention is niacin, the microencapsulates of niacin are included in an amount sufficient to provide at least about 50% niacin, preferably at least about 60%, and, in other more preferred embodiments, at least about 70% and at least about 80% in the final dosage form.

The direct compressed dosage forms of the present invention may contain at least one active agent from the above-mentioned list. However, it is contemplated that in certain embodiments, the direct compressed dosage forms may comprise a combination of two or more active agents. In certain embodiments, the direct compressed dosage forms may comprise a plurality of microencapsulated active agent substrates containing acetaminophen and a plurality of microencapsulated active agent substrates containing an opioid analgesic. Suitable opioid analgesics may include, but not limited to, alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papavereturn, pentazocine, phenadoxone, phenomorphan, phenazocine, phenoperidine, piminodine, piritramide, propheptazine, promedol, properidine, propoxyphene, sufentanil, tilidine, tramadol, tryptophan, mixtures thereof and pharmaceutically acceptable salts thereof.

### Herbal Agents

Examples of other active agents suitable for use in the present invention are herbal agents. Herbal agents may include, but are not limited to agrimony, alfalfa, allspice, angelica, anise, basil, bayberry, boneset, borage, caraway, cayenne, chamomile, dandelion, dill, Echinacea, evening primrose, fennel, garlic, ginger, gingko balboa, jasmine, juniper, lavender, lemon balm, rosemary, rue, thyme, valerian, yarrow and any other herbal that is suitable for administration to a subject/patient in need thereof. Other herbal agents suitable for use in the present invention include, but are not limited to those described in The Complete Guide to Herbal Medicines, Fetrow, Charles A., et al. (September 2000), the disclosure of which is hereby incorporated by reference.

### Nutrients

Other ingredients which may be employed as the active agent(s) in the present invention include nutritional supplements, dietary supplements and combinations thereof. The compounds meeting this criteria may have varying degrees of solubility in water ranging from highly soluble to insoluble. These compounds generally include vitamins, minerals, amino acids, herbal and botanical products and the like. Vitamins generally refer to organic substances that are required in the diet and include thiamin, riboflavin, nicotinic acid, pantothenic acid, pyrodoxine, biotin, folic acid, vitamin B12, lipoic acid, ascorbic acid (vitamin C), vitamin A, vitamin D, vitamin E and vitamin K, enzymes as well as coenzymes thereof. Minerals include inorganic substances which are required in the human diet and include calcium, iron, zinc, selenium, copper, iodine, magnesium, phosphorus, chromium, and the like and mixtures thereof.

### EXAMPLES

The present invention will be further appreciated in view of the following examples:

### Materials Used:

In the following examples soy is hydrogenated soybean oil. ProSolv® SMCC 90 is co-processed microcrystalline cellulose and colloidal silicon dioxide, an excipient, from JRS Pharma. Polyplasdone XL is crospovidone from ISP Technologies. Niacin, guaifenesin, acetaminophen (APAP), and magnesium stearate are from various sources.

### Example I

### Process for Making Soy Coated Guaifenesin Microencapsulate

12.5 lbs of guaifenesin powder was coated with 5.3 lbs of molten soybean oil using a fluidized bed encapsulation process. The molten coating was sprayed onto guaifenesin powder, at a temperature below the crystallization temperature of the soybean oil, whilst enabling the guaifenesin to be coated during fluidization. One of ordinary skill in the art will appreciate that the present invention may also be practiced utilizing other spray coating processes to provide encapsulation.

Guaifenesin microencapsulates were produced with an active agent concentration of 66.5%. Samples were also prepared at 76% and 85% concentration of active during the encapsulation process. All microencapsulates were screened to 40 mesh using a Sweco brand screener.

Other active ingredients were microencapsulated in the present examples using the same method.

### Example II

### Tableting Guaifenesin Microencapsulate

Guaifenesin microencapsulates were produced as described in Example I. 76% and 85% activity microencapsulates were used to prepare the solid dosage forms using the following formulas:

| | Type of | Tablet Formula | | | Tablet |
|---|---|---|---|---|---|
| | micro- | Micro- | Prosolv ® | Magnesium | weight |
| Tablets | encapsulates | encapsulates | SMCC 90 | stearate | (mg) |
| Tab 1 | 85% active | 80% | 19% | 1% | 877 |
| Tab 2 | 76% active | 80% | 19% | 1% | 987 |
| Tab 3 | 76% active | 90% | 9% | 1% | 877 |
| Tab 4 | 85% active | 90% | 9% | 1% | 780 |

Blending of the microencapsulates and the excipients was conducted in a Patterson-Kelly blender for ten minutes with all the ingredients except Mg-stearate, followed by two more minutes blending with Mg-stearate added to it. The blends were compressed into tablets by a Manual Tablet Compaction Machine (Model MTCM-I, GlobePharma, Inc.) at 2,000 psi. Each guaifenesin tablet contained 600 mg of guaifenesin.

### Example III

### Dissolution of Guaifenesin Tablets

Guaifenesin tablets (600 mg guaifenesin) prepared in Example II were tested for guaifenesin dissolution using a Dissolution Tester (Model VK 7000, Varian, Inc.) following USP 27/NF 22 with Apparatus II. A commercial guaifenesin extended release tablet (Mucinex® 600 mg, Adams Respiratory Therapeutics, Inc.) was also tested for comparison.

Dissolution vessels were filled with 900 ml distilled water at 37° C. The paddles were set at 50 rpm. 5 ml samples were withdrawn from each vessel at 1 hr, 2 hr, 4 hr, 6 hr, 8 hr, 12 hr, 18 hr, and 24 hr intervals. The samples were filtered through 35 μm full flow filters. The UV absorbance of each sample was measured at 274 nm to determine the dissolution of guaifenesin at each time interval. The dissolution results are presented in the table below. No disintegration of the tablets was observed during the dissolution.

% Released

| Tablets | 1 hr | 2 hr | 4 hr | 6 hr | 8 hr | 12 hr | 18 hr | 24 hr |
|---|---|---|---|---|---|---|---|---|
| Tab 1 | 32.2 | 45.9 | 64.9 | 76.7 | 87.7 | 98.3 | 100.0 | |
| Tab 2 | 23.8 | 33.5 | 47.7 | 58.5 | 67.6 | 82.1 | 93.2 | 100.0 |
| Tab 3 | 25.4 | 35.3 | 48.3 | 58.8 | 66.8 | 79.7 | 91.6 | 100.0 |
| Tab 4 | 31.7 | 45.3 | 62.0 | 74.2 | 83.0 | 94.0 | 100.0 | |
| Mucinex | 23.5 | 32.7 | 45.9 | 55.6 | 64.1 | 77.0 | 90.7 | 100.0 |

Tablets 2 and 3 had a release profile matching Mucinex. The sustained release profile for guaifenesin can be customized to different therapeutic ranges. Customization can result from changes in the type of microencapsulate (amount of coating and type of coating), the amount of microencapsulate in the tablet, and the amount and type of excipients.

### Example IV

### Tableting Acetaminophen Microencapsulate

Acetaminophen (APAP) was microencapsulated with soy to 92% active, as described in Example I, sieved through USSS 30 mesh, and the −30 mesh fraction was tableted using the following formulas:

| | Tablet Formula | | | | | |
|---|---|---|---|---|---|---|
| | APAP | Prosolv ® | Magnesium | Tablet | % APAP | Hardness |
| Tablets | Microencapsulate | SMCC 90 | Stearate | weight | in tablet | (kP) |
| A | 70% | 29% | 1% | 1020 mg | 64% | 7.9-8.2 |
| B | 60% | 39% | 1% | 1190 mg | 55% | 12.2-12.9 |

The compression mix was blended in a PK-blender for ten minutes with all the ingredients except Mg-stearate, and then two more minutes with Mg-stearate added to it. The blends were compressed into tablets by a Manual Tablet Compaction Machine (Model MTCM-I, GlobePharma, Inc.) at 2,000 psi. Tablets contained 650 mg of APAP.

### Example V

### Dissolution of Acetaminophen Tablets

The APAP tablets (650 mg of APAP) prepared in Example IV were tested for dissolution using a Dissolution Tester (Model VK 7000, Varian, Inc.) following USP 27/NF 22 with Apparatus II.

Dissolution vessels were filled with 900 ml simulated gastric fluid TS (without enzyme) at 37° C. The stir paddles were set at 50 rpm. 5 ml samples were withdrawn from each vessel at the 15 min, 1 hr, and 3 hr time intervals. The samples were filtered through 35 μm full flow filters. The UV absorbance of each sample was measured at 243 nm to determine the release of acetaminophen at each time interval. The dissolution results are presented in the table below:

% Dissolved

| Tablets | 15 min | 1 hr | 3 hr |
|---|---|---|---|
| A | 12.9 | 28.1 | 47.3 |
| B | 14.2 | 32.1 | 53.0 |

Both tablets displayed extended release. No disintegration of the tablets was observed during dissolution.

### Example VI

### Tableting Niacin Microencapsulate

Niacin was microencapsulated with soy to 88.2% activity, as described in Example II, and sieved through USSS 30 mesh, and the −30 mesh fraction was tableted using the following formula:

| | |
|---|---|
| 80% | Niacin microencapsulate |
| 19.5% | ProSolv ® SMCC 90 |
| 0.5% | Mg-stearate |

The microencapsulates and ProSolv® SMCC 90 were blended in a PK-blender for ten minutes and then two more minutes with Mg-stearate added to it. A single side 20 station press was used to make the tablets.

Acceptable tablets were made. The tablet weight was 710 mg on average; the hardness ranged from 6.5-6.7 kP. Each tablet contained 500 mg of niacin.

### Example VII

### Dissolution of Niacin Tablets

The niacin tablets prepared in Example VII were tested for dissolution using the same procedures as described in Example IV except the dissolution medium was pH 6.8 phosphate buffer. A commercial niacin extended release tablet (Niaspan® ER 500 mg, a one a day dosage e form, KOS Pharmaceuticals, Inc.) was also tested for comparison. The UV absorbance of each sample was measured at 260 nm to determine the release of niacin at each time interval. In vitro, sustained release was achieved as shown in the table below. The dissolution profile for tablets made in Example VI was similar to Niaspan®. No tablet disintegration was observed during the dissolution.

% Dissolved

| Tablets | 1 hr | 2 hr | 4 hr | 6 hr | 8 hr | 24 hr |
|---|---|---|---|---|---|---|
| Present invention | 14.9 | 23.5 | 33.8 | 42.5 | 48.7 | 80.6 |
| Niaspan | 11.0 | 17.5 | 27.3 | 36.5 | 43.9 | 91.7 |

The dissolution profile of the present invention can be modified using a number of factors as detailed in this invention to generate release over a different therapeutic range. For example, by decreasing the amount of microencapsulate in the tablet formula, a faster modified release profile, more than 80% dissolution in 12 hr (two a day dosage form), will result.

### Example VIII

### In Vivo Release of Niacin Tablets

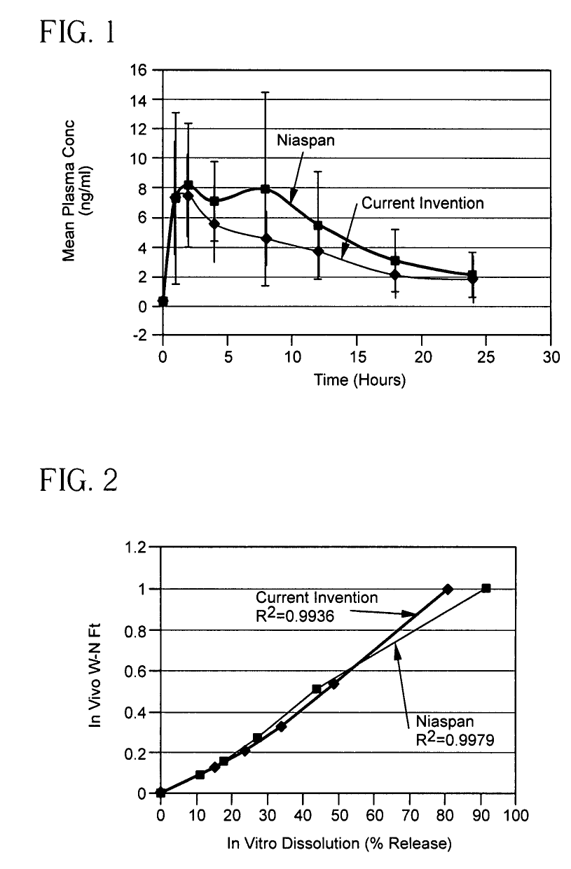

The bioavailability of niacin tablet prepared in Example VI was evaluated in a randomized, open-label pilot study with six healthy male subjects, along with the commercial niacin extended release tablet (Niaspan® ER 500 mg, a one a day dosage form, KOS Pharmaceuticals, Inc.). Blood samples were taken from the subjects at time 0 hr, 1 hr, 2 hr, 4 hr, 8 hr, 12 hr, 18 hr, and 24 hr following consumption of the dosage form. The plasma niacin concentrations are shown in FIG. 1. The plasma niacin concentrations of the current invention were comparable to those of Niaspan®.

### Correlation Study

A correlation study using the Wagner-Nelson Ft and Fx values were calculated from the Wagner-Nelson equation:

$$Ft = \frac{AUC ( t ) + Cp ( t ) / ke}{AUC ∞} . \qquad Eq . 1$$

Fx=AUC(t)+Cp(t)/ke Eq. 2

AUC(t): Area under the curve from zero to time t

AUC_{∞}: Total area under the curve

Cp(t): Plasma concentration at time t

Ke: The elimination rate constant

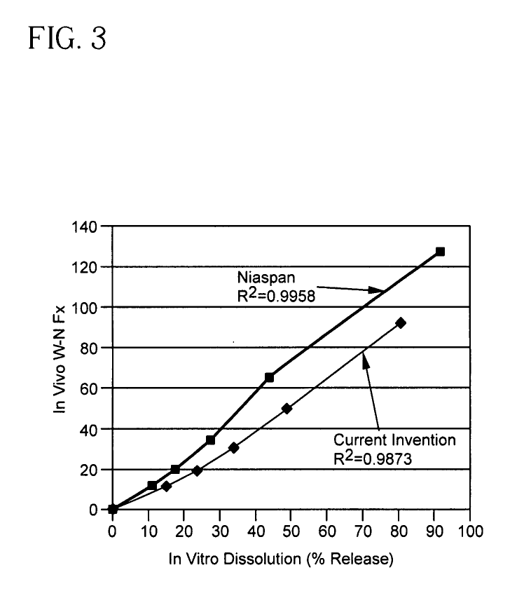

The resulting In Vivo and In Vitro Correlations (IVIVC), using Ft and Fx, are shown in FIGS. 2 and 3. Excellent IVIVC was achieved.

### Example IX

Niacin tablets containing 500 mg niacin were made using the following formula:

| | |
|---|---|
| 90% | Niacin microencapsulate (81.7% activity) |
| 9% | ProSolv ® SMCC 90 |
| 1% | Mg-stearate |

The tablets were stored under accelerated storage condition, i.e., 40° C./75% RH. The dissolution of niacin tablet over storage was measured as described in Example VII. The tablets showed excellent stability in terms of dissolution under accelerated storage conditions. No disintegration of the tablets was observed during the dissolution.

% Dissolved

| | 1 hr | 2 hr | 4 hr | 6 hr | 8 hr | 24 hr |
|---|---|---|---|---|---|---|
| Storage | Diss. | Diss. | Diss. | Diss. | Diss. | Diss. |
| 0 time | 12.2 | 19.2 | 27.8 | 34.2 | 39.3 | 63.7 |
| 5 days | 11.2 | 18.6 | 28.1 | 34.7 | 40.4 | 66.4 |
| 4 weeks | 10.0 | 16.5 | 25.7 | 32.6 | 38.2 | 66.1 |
| 16 | 9.9 | 16.4 | 25.2 | 31.8 | 36.9 | 62.6 |
| weeks | | | | | | |

### Example X

Niacin tablets were made with a disintegration agent using the following formula:

| | |
|---|---|
| 90% | Niacin microencapsulate (81.7% activity) |
| 7% | ProSolv SMCC 90 |
| 2% | Polyplasdone XL (Disintegrant) |
| 1% | Mg-stearate |

The compression mix was blended in a PK-blender for ten minutes with all the ingredients except Mg-stearate, and then two more minutes with Mg-stearate added to it. The blends were compressed into tablets by a Manual Tablet Compaction Machine (Model MTCM-I, GlobePharma, Inc.) at 2,000 psi. Each tablet contained 500 mg of niacin.

The tablets were stored under modified accelerated storage conditions, i.e., 40° C./75% RH for three (3) weeks. The dissolution of niacin tablet at day 0 and after 3 weeks storage were measured as described in Example VII. When disintegrant was included in the tablet formula, the tablets disintegrated partially during dissolution. The tablets demonstrated instability during storage. This example demonstrates that the disintegrating tablet is not part of the current invention.

% Dissolved

| Tablets | 1 hr | 2 hr | 4 hr | 6 hr | 8 hr | 24 hr |
|---|---|---|---|---|---|---|
| Day 0 | 13.1 | 20.4 | 29.3 | 36.2 | 41.4 | 68.0 |
| 40° C./75% RH for 3 weeks | 10.9 | 18.0 | 34.5 | 47.3 | 54.9 | 90.8 |

In the preceding specification, the invention has been described with reference to specific exemplary embodiments and examples thereof. It will, however, be evident that various modifications and changes may be made thereto without departing from the broader spirit and scope of the invention as set forth in the claims that follow. The examples are accordingly to be regarded in an illustrative manner rather than a restrictive sense.

What is claimed is:

1. A method of making a pharmacologically active dosage, comprising:
(a) spray coating a plurality of substrates, comprising particles including at least one pharmacologically active agent, with a neutral lipid-based coating in an amount to provide microencapsulates having an active agent concentration at least about 50%; and
(b) directly compressing said microencapsulates resulting from step (a) in an amount of at least about 50% by weight microencapsulate to form a shelf stable non-disintegrable pharmacologically active dosage unit, which releases said active agent by dissolution over the therapeutic range.

2. A method according to claim 1 wherein said microencapsulates have at least about 60% active agent by weight.

3. A method according to claim 2 wherein said active agent is present in an amount of at least about 70% by weight.

4. A method according to claim 3 wherein said active agent is present in an amount of at least about 80% by weight.

5. A method according to claim 4 wherein said active agent is present in an amount of at least about 90% by weight.

6. A method according to claim 1 wherein the level of microencapsulates in the unit dosage is at least about 60% by weight.

7. A method according to claim 6 wherein said level of microencapsulates is at least about 70% by weight.

8. A method according to claim 7 wherein said level of microencapsulates is at least about 80% by weight.

9. A method according to claim 8 wherein said level of microencapsulates is at least about 90% by weight.

10. A method according to claim 1 wherein said substrates are selected from the group consisting of (i) a granulate comprising active agent and one or more pharmaceutically acceptable diluents; (ii) active agent coated onto the surface of inert beads; and (iii) pellets comprising active agent and one or more pharmaceutically acceptable diluents.

11. A method according to claim 1 wherein said coating step is conducted by fluidized bed-coating.

12. A method according to claim 1 wherein said neutral lipid-based coating comprises a neutral lipid as a primary ingredient selected from the group consisting of triglycerides, waxes, and combinations thereof, in the substantial absence of monoglycerides and diglycerides.

13. A method according to claim 12 wherein said triglycerides are selected from the group consisting of hydrogenated vegetable oils, hydrogenated animal fats, and combinations thereof.

14. A method according to claim 13 wherein said triglyceride is a hydrogenated vegetable oil selected from the group consisting of hydrogenated soybean oil, hydrogenated palm oil, hydrogenated cottonseed oil, hydrogenated castor oil, hydrogenated canola oil, and combinations thereof.

15. A method according to claim 12 wherein said wax is selected from the group consisting of paraffin wax, carnauba wax, beeswax, candelilla wax, and combinations thereof.

16. A method according to claim 1 further comprising incorporating a hydrophobic material in said neutral lipid-based coating.

17. A method according to claim 16 wherein said hydrophobic material is ethylcellulose.

18. A method according to claim 1 further comprising an active agent in said neutral lipid-based coating prior to coating said substrates.

19. A method according to claim 1 wherein said active agent is selected from the group consisting of antihistamines; antibiotics; antituberculosis agents; cholinergic agents; antimuscarinics; sympathomimetics; sympatholytic agents; miscellaneous autonomic drugs; iron preparations; haemostatics; cardiac drugs; antihypertensive agents; vasodilators; non-steroidal anti-inflammatory agents; opiate agonists; anticonvulsants; tranquilizers; chemotherapeutic agents; lipid lowering agents; H₂-antagonists; anti-coagulant and anti-platelet agents; bronchodilators; stimulants; barbiturates; sedatives; expectorants; antiemetics; gastro-intestinal drugs; antithyroid agents; genitourinary smooth muscle relaxants; vitamins; minerals; amino acids; herbal agents; botanical agents; enzymes; unclassified agents; diabetes agents; steroids; glucocorticoids; antivirals; antifungals; antiparasitic agents; antidiabetic agents; and any combinations or mixtures of the foregoing.

20. A method according to claim 19 wherein said active agent is selected from the group consisting of acetaminophen, caffeine, guaifenesin, an opioid analgesic, ranitidine, vitamin C, potassium chloride, niacin, anti-flush agents, lipid-lowering statins, and combinations thereof.

21. A method according to claim 20 wherein said active agent comprises guaifenesin.

22. A method according to claim 21 wherein said guaifenesin is present in an amount of at least about 55% by weight of said dosage unit.

23. A method according to claim 22 wherein said guaifenesin is present in an amount of at least about 65% by weight of said dosage unit.

24. A method according to claim 23 wherein said guaifenesin is present in an amount of at least about 75% by weight of said dosage unit.

25. A method according to claim 19 wherein said active agent comprises niacin.

26. A method according to claim 25 wherein said niacin comprises at least about 50% by weight of said dosage unit.

27. A method according to claim 25 wherein said niacin comprises at least about 60% by weight of said dosage unit.

28. A method according to claim 25 wherein said niacin comprises at least about 70% by weight of said dosage unit.

29. A method according to claim 25 wherein said niacin comprises at least about 80% by weight of said dosage unit.

30. A method according to claim 19 wherein said active agent comprises niacin and at least one other lipid-lowering statin.

31. A method according to claim 19 wherein said active agent comprises niacin and at least one anti-flush agent.

32. A method according to claim 19 wherein said active agent comprises niacin and fenofibrate.

33. A method according to claim 20 wherein said active agent comprises an opioid analgesic.

34. A method according to claim 33 wherein said opioid analgesic is selected from the group consisting of codeine, hydromorphone, hydrocodone, oxycodone, morphine, meperidine and combinations thereof.

35. A method according to claim 20 wherein said active agent is acetaminophen.

36. A method according to claim 35 wherein said acetaminophen comprises at least about 50% by weight of said dosage unit.

37. A method according to claim 35 wherein said acetaminophen comprises at least about 60% by weight of said dosage unit.

38. A method according to claim 35 wherein said acetaminophen comprises at least about 70% by weight of said dosage unit.

39. A method according to claim 35 wherein said acetaminophen comprises at least about 80% by weight of said dosage unit.

40. A method according to claim 1 which further comprises adding at least one excipient ingredient prior to directly compressing.

41. A method according to claim 40 wherein said at least one excipient ingredient is included in the dosage form in an amount not to exceed 50% by weight of said dosage unit.

42. A method according to claim 41 wherein said excipient is not greater than about 40% by weight of said dosage unit.

43. A method according to claim 42 wherein said excipient is not greater than about 30% by weight of said dosage unit.

44. A method according to claim 43 wherein said excipient is not greater than about 20% by weight of said dosage unit.

45. A method according to claim 44 wherein said excipient is not greater than about 10% by weight of said dosage unit.

46. A method according to claim 45 wherein said excipient is not greater than about 5% by weight of said dosage unit.

47. A method according to claim 40 wherein said excipient is selected from the group consisting of flavoring agents, acidifiers, sweeteners, taste-maskers, lubricants and combinations thereof.

48. A method according to claim 40 wherein said excipient comprises an ingredient selected from the group consisting of sucrose, dextrose, lactose, microcrystalline cellulose, xylitol, fructose, sorbitol, maltodextrin, polyethylene glycol (PEG), silicified microcrystalline cellulose and combinations thereof.

49. A method according to claim 1 wherein said active dosage unit active agent dissolves from the active dosage form in the absence of disintegration during dissolution of active within the therapeutic range.

50. A method according to claim 49 wherein said active agent is guaifenesin and said dosage unit is designed to release from about 10% to about 35% of said guaifenesin after one (1) hour and from about 50% to about 90% after eight (8) hours in vitro.

51. A method according to claim 49 wherein said active agent is niacin in a one a day dosage unit wherein said unit is designed to release from about 5% to about 30% of said niacin after one (1) hour and from about 35% to about 70% after eight (8) hours in vitro.

52. A method according to claim 49 wherein said active agent is niacin in a two a day dosage unit wherein said unit is designed to release from about 5% to about 40% of said niacin after one (1) hour and from about 25% to about 75% after four (4) hours in vitro.

53. A method according to claim 1 wherein said dosage unit has an active agent “early and sustained” modified release.

54. A method according to claim 1 wherein said dosage unit has an active agent release which is one of delayed, controlled, extended, site specific, slow, pulsatile, modified, and combinations thereof.

55. A pharmacologically active dosage unit comprising:
a compressed non-disintegrable tablet comprising a plurality of substrates microencapsulated in a neutral lipid-based coating, said substrates comprising at least one pharmacologically active ingredient in an amount to provide a concentration of active agent of at least about 50% in said microencapsulate, and said microencapsulate included in said dosage unit in an amount of at least 50% by weight of said dosage unit such that said active ingredient exhibits modified release by dissolution throughout the therapeutic range prescribed for treating a patient in need of said active ingredient.

56. A dosage unit according to claim 55 wherein said concentration of said active agent in said microencapsulate is at least about 60% by weight.

57. A dosage unit according to claim 56 wherein said concentration of said active agent is at least about 70% by weight.

58. A dosage unit according to claim 57 wherein said concentration of said active agent is at least about 80% by weight.

59. A dosage unit according to claim 58 wherein said concentration of said active agent is at least about 90% by weight.

60. A dosage unit according to claim 55 wherein the level of microencapsulate in said dosage unit is at least about 60% by weight.

61. A dosage unit according to claim 60 wherein said level of microencapsulate is at least about 70% by weight.

62. A dosage unit according to claim 61 wherein said level of microencapsulate is at least about 80% by weight.

63. A dosage unit according to claim 62 wherein said level of microencapsulate is at least about 90% by weight.

64. A dosage unit according to claim 55 wherein said substrates are selected from the group consisting of (i) a granulate comprising active agent and one or more pharmaceutically acceptable diluents; (ii) active agent coated onto the surface of inert beads; and (iii) pellets comprising active agent and one or more pharmaceutically acceptable diluents.

65. A dosage unit according to claim 55 wherein said neutral lipid-based coating comprises a neutral lipid as a primary ingredient selected from the group consisting of triglycerides, waxes, and combinations thereof, in the substantial absence of monoglycerides and diglycerides.

66. A dosage unit according to claim 55 wherein said triglyceride is selected from the group consisting of hydrogenated vegetable oil, hydrogenated animal fat, and combinations or mixtures thereof.

67. A dosage unit according to claim 66 wherein said triglyceride is a hydrogenated vegetable oil selected from the group consisting of hydrogenated soybean oil, hydrogenated palm oil, hydrogenated cottonseed oil, hydrogenated castor oil, hydrogenated canola oil, and combinations thereof.

68. A dosage unit according to claim 65 wherein said wax is selected from the group consisting of paraffin wax, carnauba wax, beeswax, candelilla wax, and combinations thereof.

69. A dosage unit according to claim 55 wherein said neutral lipid-based coating further comprises a hydrophobic material.

70. A dosage unit according to claim 69 wherein said hydrophobic material is ethylcellulose.

71. A dosage unit according to claim 55 wherein said active agent is selected from the group consisting of antihistamines; antibiotics; antituberculosis agents; cholinergic agents; antimuscarinics; sympathomimetics; sympatholytic agents; miscellaneous autonomic drugs; iron preparations; haemostatics; cardiac drugs; antihypertensive agents; vasodilators; non-steroidal anti-inflammatory agents; opiate agonists; anticonvulsants; tranquilizers; chemotherapeutic agents; lipid lowering agents; H₂-antagonists; anti-coagulant and anti-platelet agents; bronchodilators; stimulants; barbiturates; sedatives; expectorants; antiemetics; gastro-intestinal drugs; antithyroid agents; genitourinary smooth muscle relaxants; vitamins; minerals; amino acids; herbal agents; botanical agents; enzymes; unclassified agents; diabetes agents; steroids; glucocorticoids; antivirals; antifungals; antiparasitic agents; antidiabetic agents; and any combinations or mixtures of the foregoing.

72. A dosage unit according to claim 71 wherein said active agent is selected from the group consisting of acetaminophen, caffeine, guaifenesin, an opioid analgesic, ranitidine, vitamin C, potassium chloride, lipid-lowering statins, niacin, anti-flush agents, and combinations thereof.

73. A dosage unit according to claim 72 wherein the active agent is acetaminophen.

74. A dosage unit according to claim 73 wherein said acetaminophen comprises at least about 50% by weight of said dosage unit.

75. A dosage unit according to claim 73 wherein said acetaminophen comprises at least about 60% by weight of said dosage unit.

76. A dosage unit according to claim 73 wherein said acetaminophen comprises at least about 70% by weight of said dosage unit.

77. A dosage unit according to claim 73 wherein said acetaminophen comprises at least about 80% by weight of said dosage unit.

78. A dosage unit according to claim 55 which further comprises a second active agent.

79. A dosage unit according to claim 78 wherein said additional active agent is an opioid analgesic.

80. A dosage unit according to claim 79 wherein said opioid analgesic is selected from the group consisting of codeine, hydromorphone, hydrocodone, oxycodone, morphine, meperidine and any combination of the foregoing.

81. A dosage unit according to claim 55 which further comprises at least one excipient mixed with the microencapsulated substrates.

82. A dosage unit according to claim 81 wherein said at least one excipient ingredient is included in the dosage form in an amount not to exceed 50% by weight of said dosage unit.

83. A dosage unit according to claim 82 wherein said excipient is not greater than about 40% by weight of said dosage unit.

84. A dosage unit according to claim 83 wherein said excipient is not greater than about 30% by weight of said dosage unit.

85. A dosage unit according to claim 84 wherein said excipient is not greater than about 20% by weight of said dosage unit.

86. A dosage unit according to claim 85 wherein said excipient is not greater than about 10% by weight of said dosage unit.

87. A dosage unit according to claim 86 wherein said excipient is not greater than about 5% by weight of said dosage unit.

88. A dosage unit according to claim 81 wherein said excipient is selected from the group consisting of flavoring agents, acidifiers, sweeteners, taste-maskers, lubricants and any combinations or mixtures of the foregoing.

89. A dosage unit according to claim 81 wherein said excipient is selected from the group consisting of sucrose, dextrose, lactose, microcrystalline cellulose, xylitol, fructose, sorbitol, maltodextrin, polyethylene glycol (PEG), silicified microcrystalline cellulose, and combinations thereof.

90. A dosage unit according to claim 71 wherein said active agent comprises guaifenesin.

91. A dosage unit according to claim 90 wherein said guaifenesin is present in an amount of at least about 55% by weight of said dosage unit.

92. A dosage unit according to claim 91 wherein said guaifenesin is present in an amount of at least about 65% by weight of said dosage unit.

93. A dosage unit according to claim 92 wherein said guaifenesin is present in an amount of at least about 75% by weight of said dosage unit.

94. A dosage unit according to claim 71 where said active agent comprises niacin.

95. A dosage unit according to claim 94 wherein said niacin comprises at least about 50% by weight of said dosage unit.

96. A dosage unit according to claim 94 wherein said niacin comprises at least about 60% by weight of said dosage unit.

97. A dosage unit according to claim 94 wherein said niacin comprises at least about 70% by weight of said dosage unit.

98. A dosage unit according to claim 94 wherein said niacin comprises at least about 80% by weight of said dosage unit.

99. A dosage unit according to claim 71 wherein said active agent comprises niacin and at least one other lipid-lowering statin.

100. A dosage unit according to claim 71 wherein said active agent comprises niacin and at least one anti-flush agent.

101. A dosage unit according to claim 71 wherein said active ingredient comprises niacin and fenofibrate.

102. A dosage unit according to claim 55 wherein said active dosage unit active agent dissolves in the absence of disintegration during dissolution of active within the therapeutic range.

103. A dosage unit according to claim 102 wherein said active is guaifenesin and said dosage unit is designed to release from about 10% to about 35% of said guaifenesin after one (1) hour and from about 50% to about 90% after eight (8) hours in vitro.

104. A dosage unit according to claim 102 wherein said active is niacin in a one a day dosage unit wherein said unit is designed to release from about 5% to about 30% of said niacin after one (1) hour and from about 35% to about 70% after eight (8) hours in vitro.

105. A dosage unit according to claim 102 wherein said active is niacin in a two a day dosage unit wherein said unit is designed to release from about 5% to about 40% of said niacin after one (1) hour and from about 25% to about 75% after four (4) hours in vitro.

106. A dosage unit according to claim 55 wherein said dosage unit has an active agent “early and sustained” modified release.

107. A dosage unit according to claim 55 wherein said dosage unit has an active agent release which is one of delayed, controlled, extended, site specific, slow, pulsatile, modified, and combinations thereof.

108. A pharmacologically active dosage unit comprising:
a compressed non-disintegrable tablet comprising a plurality of substrates microencapsulated in a neutral lipid-based coating, said substrates comprising at least one pharmacologically active ingredient in an amount to provide a concentration of active agent of at least about 50% in said microencapsulate, and said microencapsulate included in a one-component dosage unit in an amount of at least 50% by weight of said dosage unit such that said active ingredient exhibits modified release by dissolution throughout the therapeutic range prescribed for treating a patient in need of said active ingredient.
